(19) United States
(12) Patent Application Publication
(10) Pub. No.: US 2018/0296128 A1
(43) Pub. Date: Oct. 18, 2018

(54) ACTIVE ORTHOSIS SYSTEM

(71) Applicant: Yves Crimersmois, Boissy Saint Leger (FR)

(72) Inventors: Hugo CRIMERSMOIS, Boissy Saint Leger (FR); Ming SHI, Evry (FR); Matthieu CRIMERMOIS, Boissy Saint Leger (FR); Yves CRIMERMOIS, Boissy Saint Leger (FR)

(21) Appl. No.: 15/762,886

(22) PCT Filed: Sep. 26, 2016
(86) PCT No.: PCT/EP2016/072814

§ 371 Date: Mar. 23, 2018

(30) Foreign Application Priority Data
Sep. 25, 2015 (FR) 1559053

(51) Int. Cl. A61B 5/107; A61B 5/11; A61B 5/00; A61D 19/02

(52) U.S. Cl. CPC A61B 5/1071; A61B 5/1126; A61B 5/744; A61B 5/746; A61B 5/1121; A61D 19/02; A61B 2562/0219; A61B 5/6823; A61B 5/6824; A61B 5/1116

(57) ABSTRACT

The invention relates to an active orthosis system comprising a plurality of inertial sensors to be distributed over an upper limb, a lower limb, or any other part of the human body comprising at least one joint, said inertial sensors (1, 2, 3, 4) being designed so as to allow the determination of at least one angle formed by segments of said part of the human body, around said at least one joint. The invention is characterised in that said active orthosis system comprises an alert device for warning the user of the active orthosis system that said at least one angle has a value located outside a pre-determined range of comfort values.

### CROSS-REFERENCE TO RELATED APPLICATIONS

This application is entitled to the benefit of and incorporates by reference subject matter disclosed in the International Patent Application No. PCT/EP2016/072814 filed on Sep. 26, 2016 and French Patent Application 1559053 filed Sep. 25, 2015.

### TECHNICAL FIELD

The present invention generally relates to assisting professionals whose occupation involves holding postures, making repetitive movements or blind movements, risking causing joint traumas. More specifically, the present invention aims for an active orthosis system having the aim of helping these professionals to optimise their working postures such that the angular degrees of their joints remain in “comfort zones”, which do not cause trauma for said joints.

Many professions really call for the application of professionals' joints, to the point of being able to cause traumas and injuries inducing work interruptions for health reasons and sometimes, surgical work to repair these traumas and injuries. A specific example resides in carrying out the occupation of a bovine artificial insemination technician.

One of the specifics of this occupation is that artificial insemination technicians carry out the act of insemination without being able to directly see the movements that they are making inside cows to inseminate them. For the technician, the only criteria to evaluate the effectiveness of their movement resides in the success of the act of insemination in itself. Indeed, the technician today is not able to consider movements and postures adopted by their arm to succeed in this act.

The consequences on the health of insemination technicians are frequent and can be very serious. Indeed, one insemination technician out of two on average has their rotator cuff operated on at the end of 20 years to 25 years of professional practice.

Other professions are concerned by similar problems, because of the need to make repetitive movements and/or blind movements, or again, to adopt specific postures. Another example of an occupation wherein professionals can find themselves faced with joint problems is that of a checkout assistant, in a supermarket. Repeated movements to make a product go from one side of the checkout to the other, can lead to joint pains and traumas, at the level of the wrists, elbows or shoulders, or again, problems at the level of the spine or cervical vertebrae.

No satisfactory technical solution exists, in the state of the art, to allow the measurement in real time of the state of a movement or of a posture in view of alerting a technician who dangerously applies a joint, in order to allow them to correct the movement or the posture.

It is only known, today, to define “comfort zones” consisting of determining ranges of comfort values, inside which it is recommended to hold angles formed at the level of the joints, in order to minimise the risk of injuries and traumas. Through training and practice sessions, technicians can be trained to adopt movements and postures allowing them to learn how to hold angles formed at the level of their joints inside said predetermined ranges of values. Such training however is not enough, in particular because, in real time, there is generally a difference between the movement that the technician thinks they are making, and the movement they actually make, their movements being made blindly.

Because of this, it is common that technicians called upon to make repeated movements, in particular, with variable or stable but rapid angular speeds, suffer from Musculoskeletal Disorders, known under the acronym of MSD. MSDs are lesions acquired, following repeated traumas, which mainly sit at the level of the joints. These problems are also qualified as periarticular diseases, and affect, for example, the wrists, elbows, shoulders, knees and intervertebral joints, etc. These are mainly work-related diseases.

Therefore, a need has appeared for a tool allowing gestures and movements of bovine artificial insemination technicians to be educated, such that they can carry out the act of insemination, while avoiding pejorative postures for their joints. Yet, in work physiology, comfort zones regarding movements and postures have truly been defined and allow to preserve the osteoarticular health of technicians, and thus prevent Musculoskeletal Disorders with upper limbs. For example, it is recommended to not repeatedly extend the wrist more than 30 degrees.

In this context, a first approach could consist of designing a sort of orthosis or articulated exoskeleton having stoppers to prevent the technician adopting movements and postures making angles formed at the level of their joints from predetermined comfort zones. However, this “rigid” solution cannot be considered in many applications, as it is too cumbersome and uncomfortable, in particular, for artificial insemination technicians. Thus, this device impedes the activity of operators and no longer allows them to carry out their work under satisfactory conditions.

Therefore, there is truly a need for an alert system, functioning in real time, allowing to warn a technician who is in the process of applying their joints dangerously, and giving them means to correct their movement or their posture.

In this context, the present invention arises from the idea of developing a tool allowing to reconstitute the possibility of a visualisation, by the technician, of the movements and postures adopted, through the intermediary of an active orthosis system, worn by said technician, and of their digital avatar, corresponding to a virtual representation on a screen of the movements and postures adopted by the technician, in real time.

The artificial insemination technician, for example, therefore wears the active orthosis and carries out the act of insemination, while the digital avatar makes the same movements in real time and adopts the same postures as the technician. Subsequently, through using the active orthosis system according to the invention, the success criteria of the insemination movement no longer only correspond to the success of the insemination as such but resides in the success of the insemination with a movement made in the joint comfort zone indicated by the active orthosis.

### SUMMARY

To this end, the invention aims for an active orthosis system, comprising a plurality of inertial sensors configured to be distributed over an upper limb, a lower limb, or any other part of the human body comprising at least one joint. The active orthosis system according to the invention is particularly remarkable in that said inertial sensors are designed to allow the determination of at least one angle formed by segments of said part of the human body, around said at least one joint, and in that the active orthosis system comprises an alert device for warning the user of the active orthosis system that said at least one angle has a value located outside of a predetermined range of comfort values.

This active orthosis system, in particular, allows an operator implementing it to identify the movements and the postures to avoid. Through the intermediary of the alert device, the operator knows if they are making movements and postures which do not damage their joints, or if, on the contrary, they are making movements and postures that are pejorative for their joints.

According to a preferred embodiment, the active orthosis system comprises software means to represent on a computer screen, at least said part of the human body, in the form of an avatar, presenting, in real time, said at least one angle of said at least one joint, such as determined.

Advantageously, the active orthosis system can comprise means for presenting to the user, the visual and/or sound information adapted to lead them to correct their posture so as to hold said at least one angle in the predetermined range of comfort values.

According to an embodiment, the active orthosis system comprises means for generating vibrations felt by the user when said at least one angle exits the predetermined range of comfort values.

Advantageously, the active orthosis system can comprise wireless communication means, for example, conform with the Bluetooth standard, so as to communicate data from the plurality of sensors to a calculator or so as to communicate information to the user.

Advantageously, the plurality of inertial sensors comprises at least one sensor of at least one of the following types: accelerometer; gyroscope; magnetometer.

In a preferred manner, each inertial sensor from the plurality of inertial sensors consists of a nine-axis detection module, together comprising a three-axis accelerometer, a three-axis gyroscope and a three-axis magnetometer.

Advantageously, the active orthosis system comprises a microcontroller.

According to a specific application of the orthosis system according to the invention, the part of the human body is an upper limb, the active orthosis system comprising four inertial sensors arranged respectively on the middle of the arm, on the middle of the forearm, on the back of the hand and at the level of the plexus, allowing to determine the angles of the shoulder, the elbow and the wrist.

Moreover, the active orthosis system according to the invention can advantageously comprise software means capable of transferring a set of parameters to the active orthosis system, as well as collecting and processing information from the plurality of inertial sensors statistically.

The results of processing said information by said software means allow, in particular, the representation of this information in graphic form, intended for a user, as well as an interpretation of said information, comprising, for example, information about the number of times where the angular degree of a joint comes out of the range of comfort values. This information and the interpretation thereof can be presented so as to be able to be seen by a user on a computer screen, for example. In this case, the active orthosis system constitutes a tool for measuring movements, and the developed software means form a communication interface between the user and the active orthosis system implemented.

In addition, the present invention also aims for a method for determining a recommended value for at least two angles corresponding to at least two joints of a part of the human body, such as an upper limb, according to the determination, by an active orthosis system, such as briefly defined above, of said at least two angles formed by the segments of said part of the human body, respectively around each one of said at least two joints.

### BRIEF DESCRIPTION OF THE DRAWINGS

The invention will be best understood upon reading the description which follows, given only as an example, and by referring to the appended drawings, whereon:

FIG. 1 corresponds to the schematic representation of an example of a material design of the active orthosis system according to the invention;

FIG. 2A shows the definition of the angles of the shoulder;

FIG. 2B shows the definition of the angles of the elbow;

FIG. 2C shows the definition of the angles of the wrist;

FIG. 3 shows the arrangement of the plurality of inertial sensors around an upper limb.

It must be noted, that the description which follows defines the invention in a detailed manner, the figures could, of course, be used to best define the invention, if necessary.

### DETAILED DESCRIPTION

In what follows, the active orthosis system according to the invention is more specifically defined in the context of an implementation in the field of bovine artificial insemination. The active orthosis is thus defined, consequently, as being worn by an animal insemination technician. Wearing the active orthosis system according to the invention at the level of the arm will have the function of helping said technician to adopt the suitable posture and movements in order to avoid the occurrence of joint problems at the level of the shoulder, the elbow or the wrist.

However, the whole implementation of the active orthosis system according to the invention in a different context is also covered by the present invention. As soon as repeated movements and/or movements made blindly, or delicate postures, are necessary, wearing the active orthosis system according to the invention allows to alert the operator during movements or postures which apply one of their joints incorrectly. For example, an implementation of the active orthosis system according to the invention for a use by checkout assistants in a supermarket is particularly considered and aimed for by the present invention.

Furthermore, using the active orthosis system according to the invention, and in particular, the statistical interpretation of data from the use thereof, moreover allow the operator to feedback on their experience and to be optimally trained.

Theoretically, in order to define the angular movements of a rigid body in the space, the posture of this body can be estimated by the mixed formalism of Euler angles and quaternions.

One of the difficulties resides in estimating the state of a dynamic system, typically the orientation in the space of a moving body, from a series of potentially incomplete or noisy measurements, from very different origins and types.

In the framework of the present invention, a method of detection with a recursive filtering algorithm has therefore been highlighted, based on a principle of merging data, for determining the posture in the space of a rigid body in real time. This method of determining the posture in the space of a rigid body in real time is detailed later.

The latter has the aim, in fine, of determining the angular degree of the joints followed by the active orthosis system, in the application considered. The posture in the space of a rigid body, which must be determined in real time, in view of determining the angle formed followed by a joint of the human body (typically the wrist, the elbow, or the shoulder) is achieved through using a plurality of inertial sensors, of the accelerometer/gyrometer/magnetometer type, allowing a movement capture along nine axes, in order to detect the vectors corresponding to the North and the Earth's gravity in relation to identifying the sensors taking these measurements.

### Example of a Material Design

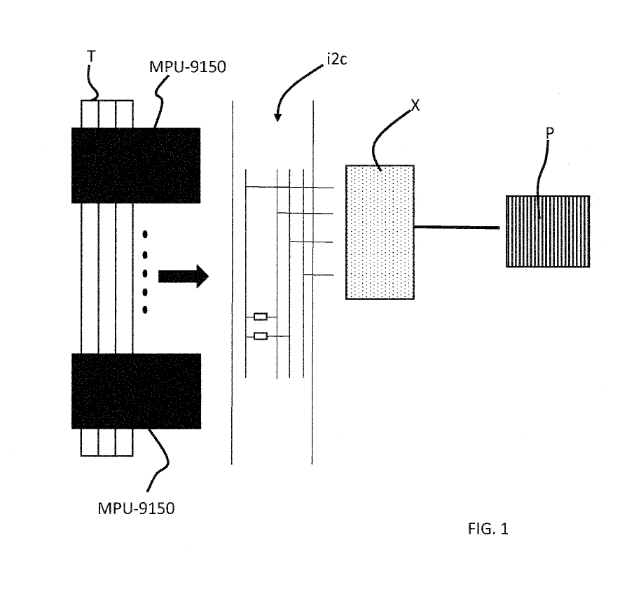

In reference to FIG. 1, an example of material architecture of the active orthosis system according to the invention is presented.

### Sensors

In an example of an embodiment of the active orthosis system according to the invention, the inertial sensors used are MPU-9150 sensors, proposed by the company InvenSense. The MPU-9150 is a module allowing to detect the orientation over nine degrees of freedom. It is composed of an MPU-6050 sensor, which comprises a three-axis gyroscope and a three-axis accelerometer, and an AK 8975 sensor, which is a three-axis magnetometer. The three-axis accelerometer measures the linear acceleration along three axes of a direct orthogonal system x, y and z. In concrete terms, the connected measurement is given in g. The three-axis gyrometer measures the angular speed around said three axes x, y and z. The measurement is taken in 7 s. The three-axis magnetometer measures the magnetic field, always over three axes x, y and z. The measurement is given in μT. The many accessible registers allow to design the MPU-9150 to the needs of the application, whether in terms of precision of the gyroscope, accelerometer, or magnetometer.

It can, of course, be considered to use any other sensor reference, allowing to determine their posture in the space in real time, preferably along nine axes.

### Microcontroller

On a material level, according to a preferred embodiment, the active orthosis system according to the invention, moreover comprises a microcontroller P, in particular capable of making calculations necessary for the processing of data from these sensors.

Moreover, it can be noted, that according to a preferred embodiment, the system implements a communication bus that conforms with the i2c protocol.

Furthermore, the active orthosis system according to the invention implements a multiplexor X capable of ensuring multiplexing, on the communication bus, of data from the plurality of sensors.

Typically, it is considered, in the framework of an active orthosis adapted to be worn on the arm of an animal insemination technician, to have four inertial sensors distributed over the arm, to capture in real time the angles of all the joints of the upper limb of the body. The adapted multiplexing means X are consequently provided to allow the flow of data from these different sensors onto the communication bus.

Preferably, an expansion card can also be provided in order to reduce the volume of the device.

Finally, according to an embodiment, conductive tape with four channels can be implemented and inserted into a sleeve covering the arm (or any other part of the body comprising at least one joint to be monitored), thus forming an electrical cabling T allowing the implementation of the system. These four conductors, for example, made of soft silver, are preferably insulated in a polyester strip before being inserted.

An adapted casing and supply are also provided, their choice being in the scope of a person skilled in the art.

### Implementation of the Active Orthosis System for an Upper Limb of the Human Body (Arm)

### Angles

In this part, calculation formalisms are defined for angles of joints of the upper limb through an anatomic approach, by using different mobile unit vectors for each inertial sensor, said mobile unit vectors being expressed in laboratory standards.

It must be noted that, through measuring angles, the orthosis system allows, not only to determine the value of said angles in degrees, but also the angular speeds and the frequency of the movements made by the joints corresponding to said angles.

### Shoulder:

The shoulder is articulated between the scapula (blade) and the humerus. This joint forms part of the shoulder girdle.

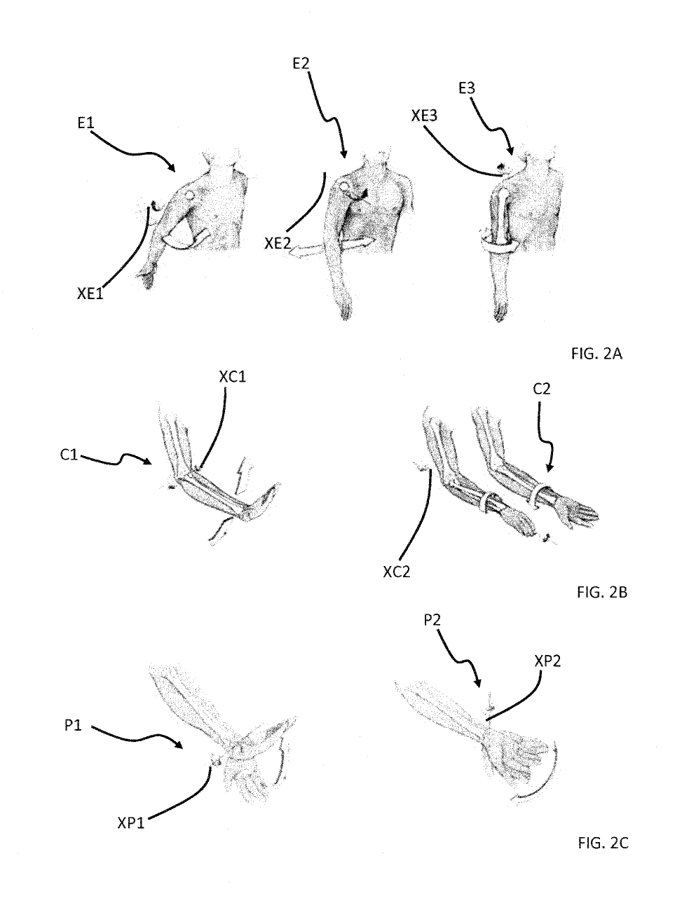

FIG. 2A shows the different degrees of freedom of this limb and the three angles which can, for example, be used to model the movements of these joints. The first shoulder angle E1 defines the rotation around the transverse axis XE1, as extension and flexion. The second shoulder angle E2 defines the rotation around the sagittal axis XE2, in particular allowing to define two movements: adduction and abduction. Finally, the third shoulder angle E3 defines the rotation around the longitudinal axis XE3, via two angular movements: internal rotation (medical rotation) and external rotation (lateral rotation).

### Elbow:

The elbow articulates between the humerus on the one hand, and the ulna (cubitus) and the radius on the other hand.

In reference to FIG. 2B, there are, at the level of the elbow, two degrees of freedom around the transverse axis XC1 as flexion and extension, conveyed through the intermediary of the first elbow angle C1. The second elbow angle C2 allows to represent pronation and supination, around the rotation axis XC2, as represented in FIG. 2B.

### Wrist:

The hand articulates between the ulna (cubitus) and the radius on the one hand, and the carpal bones on the other hand.

At the level of the wrist, flexion and extension are defined using the first wrist angle P1, around the transverse axis XP1, and abduction and adduction are defined through the intermediary of the second wrist angle P2, around the sagittal axis XP2, as is represented in FIG. 2C.

To calculate the seven angles E1, E2, E3, C1, C2, P1, P2 of the upper limb in laboratory standards, a plurality of inertial sensors 1, 2, 3, 4 is distributed adaptively for this part of the human body.

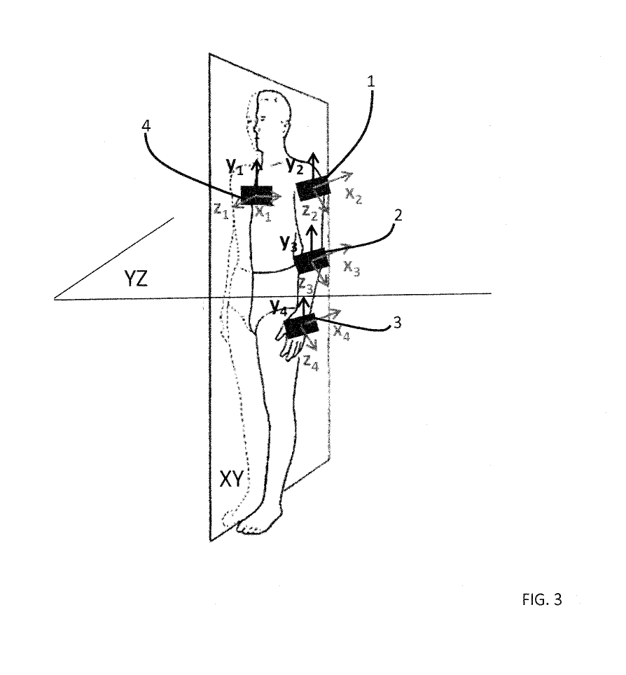

Each sensor 1, 2, 3, 4 is thus arranged, according to a preferred embodiment, on either side of each joint (shoulder, elbow, wrist), as represented in FIG. 3.

At rest, the axes of the sensors 1, 2, 3, 4 are aligned with the anatomic axes: sagittal axis X, anteroposterior axis Y and mediolateral axis Z.

Moreover, still in reference to FIG. 3, the unit vectors x₁, y₁, z₁ correspond to the unit vectors of the direct orthonormal system connected to the sensor 1 and expressed in the laboratory system. Similarly, the unit vectors x₂, y₂, z₂ correspond to the unit vectors of the direct orthonormal system connected to the sensor 2 and expressed in the laboratory system, the unit vectors x₃, y₃, z₃ correspond to the unit vectors of the direct orthonormal system connected to the sensor 3 and expressed in the laboratory system, and the unit vectors x₄, y₄, z₄ correspond to the unit vectors of the direct orthonormal system connected to the sensor 4 and expressed in the laboratory system.

Thus, the orientation of the sensor 2 in relation to the reference sensor, in other words sensor 1, allows to calculate the three shoulder angles. In reference to FIG. 3, the first shoulder angle E1 is easily obtained:

E₁=π−a cos (\vec{y}₁·\vec{y}₂) (1)

\vec{y}₁ and \vec{y}₂ being expressed in the laboratory system, the positive value gives the flexion movement and the negative value defines the extension movement.

The second shoulder angle E2 can thus be obtained indirectly, via two vectoral products:

E₂=a cos (((\vec{y}₁Λ\vec{y}₂)Λ\vec{y}₁)·\vec{z}₁) (2)

To separate the abduction and adduction movements, a sign convention is defined:

Si ((\vec{y}₁Λ\vec{y}₂)Λ\vec{y}₁)·\vec{x}₁>0, E₂=−E₂ (3)

The third shoulder angle E3 corresponds to the angle between two vectors \vec{x}₁ and \vec{z}₂^{Rot} where \vec{z}₂^{Rot} is the new vector z₂ after consecutive rotations connected to the first shoulder angle E₂ around the axis y₁ and to the first shoulder angle E₁ around the axis x₁. This term is calculated by applying the Rodrigues rotation formula, which states that, for any vector \vec{U}, a vector \vec{V} can be noted as being the image of the vector \vec{U} by the rotation (\vec{N}, φ), so:

\vec{V}=(cos ϕ)\vec{U}+(1+cos ϕ)(\vec{U}·\vec{N})\vec{N}+(sin ϕ)(\vec{N}Λ\vec{U})

From which it arises that, for the second shoulder angle E2:

\vec{z}₂^{tmp}=(cos E₂)\vec{z}₂+(1+cos E₂)(\vec{z}₂·\vec{y}₁)\vec{y}₁+(sin E₂)(\vec{y}₁Λ\vec{z}₂)

\vec{z}₂^{Rot}=(cos E₁)\vec{z}₂^{tmp}+(1+cos E₁)(\vec{z}₂^{tmp}·\vec{x}₁)\vec{x}₁+(sin E₁)(\vec{x}₁Λ\vec{z}₂^{tmp}) (4)

The third shoulder angle E3 is thus expressed as:

E₃=π−a cos(\vec{z}₂^{Rot}·\vec{x}₁) (2)

The sign convention to distinguish the internal rotation (positive) and external rotation (negative) here is put forward as follows:

Si \vec{z}₂^{Rot}·\vec{z}₁>0, E₃=−E₃ (6)

The same methodology can be used to calculate the elbow angles and the wrist angles. The orientation of the sensor 3 in relation to the sensor 2, will give the two elbow angles. The first elbow angle C1 is obtained by:

C₁=a cos(\vec{y}₂·\vec{y}₃) (7)

The second elbow angle C2 is calculated through the intermediary of two vectors \vec{x}₂ and \vec{z}₃^{Rot} where \vec{z}₃^{Rot} is the new vector z₃ after a rotation connected to the first elbow angle C1 around the axis z₂. By application, as before, of the Rodrigues rotation formula:

\vec{z}₃^{Rot}=(cos C₁)\vec{z}₃+(1+cos C₁)(\vec{z}₃·\vec{z}₂)\vec{z}₂−(sin C₁)(\vec{z}₂Λ\vec{z}₃) (8)

The second elbow angle C2 is equal to:

C₂=−a cos(\vec{z}₂·\vec{z}₃^{Rot})+π/2 (9)

Using the relative orientation of the sensor 3 in relation to the sensor 4, the wrist angles P1, P2 can be obtained directly by calculating the scalar products:

P₁=a cos(\vec{y}₃·\vec{z}₄)−π/2 (10)

And

P₂=−a cos(\vec{y}₃·\vec{x}₄)+π/2 (11)

Furthermore, possible disturbances interfering with measurements taken using the inertial sensors 1, 2, 3, 4 can, if necessary, form the subject of corrective measures by specific electronic and software means.

Through the intermediary of the preceding equations, it is consequently made possible to access the values, in real time, of the angles E1, E2, E3, C1, C2, P1, P2 of the joints of the upper limb, by using the measurements of a system distributed with inertial sensors 1, 2, 3, 4.

Using the active orthosis system according to the invention is further provided to be optimised by means of using an adapted software, allowing to make embedded calculations, in real time, and to store said values in a memory space, if necessary.

In particular, the standard C++ libraries can be used to access data from inertial sensors and communicate on a databus via the communication protocol i2c.

Thus, once the calculation of the angles E1, E2, E3, C1, C2, P1, P2 of the joints of the upper limb (shoulder, elbow, wrist) is made, these values can be sent in real time on a communication bus connected to a network connected to a workstation.

As a reminder, the values corresponding to said angles can thus truly be measurements of angles per se, in degrees, angular speeds, from the development over time of the value of said angles, or the frequency or repetitive measurements of a given movement.

On the screen of this workstation, according to a preferred embodiment, an avatar is displayed, representing all or part of a person corresponding to the operator using the active orthosis. The values of the angles of the joints, such as calculated, allow an update in real time of the same angles on the avatar displayed on the screen.

This avatar can, for example, be created in OpenGL ES.

A save phase can preferably be provided. The successive values of the angles E1, E2, E3, C1, C2, P1, P2 of the joints of the upper limb can, for example, be stored in a text file. The recording frequency of saving data can typically be around 25 Hz, allowing a later interpretation in the form of a video reconstitution.

According to an embodiment, the frequency of calculating the angles and interpreting the values for updating the avatar is around 100 Hz.

With the possibility to display in real time, via an avatar, the movements made by the technician constitutes a great advantage, in particular allowing to supply a direct view of the movements achieved.

In addition, recording the data at a frequency of around 25 Hz allows a fluid movement during a video reconstitution of the technician's performance, for analysis and statistical interpretation purposes, for example.

### Use of Data

### Alert Means

According to a preferred embodiment of the active orthosis system according to the invention, visual and/or sound alert means are triggered as soon as at least one of the angles of the joints exceed a certain angular degree, in other words, as soon as it exits a predetermined range of comfort values, said values could be configurable.

According to another embodiment, the active orthosis system can also comprise means for generating vibrations directly on the part of the body, so as to alert the operator by these vibrations.

Indeed, in work physiology, the comfort zones regarding movements and postures are known as allow to preserve physiological health. If the degree of angulation of the joints induced by a movement exceeds the degree of the comfort zone, this movement is considered as damaging for the joint. As soon as the angle of the joint exceed the angle of the comfort zone, the alert system is triggered. The operator wearing the active orthosis is consequently prompted to correct their posture, to rectify their movement, so as to bring all the angles back into the comfort zone, in other words, in the predetermined range of values considered as safe from a physiological point of view.

Different means in the scope of a person skilled in the art can be implemented to produce said alert means. For a visual perception of the alert trigger, LEDs can be installed on the active orthosis system. Moreover, warning lights or specific messages can be displayed on the computer screen. For example, when the hand flexes too much, the hand of the avatar is displayed in red.

For an auditive perception, the emission of a sound signal or a specific vocal message can be provided. For example, according to a specific embodiment, the installation of piezoelectric buzzers is provided.

A tactile perception of the alert trigger can also be implemented, according to another embodiment, through the intermediary of means capable of generating vibrations, installed on the active orthosis in contact with the body.

In a preferred manner, the different alert means implemented are adapted to give the operator information to lead them, if necessary, to correct their posture so as to hold each one of the angles adopted by their joints in the predetermined ranges of comfort values.

According to the invention, the results of the measurements and the calculations of angles of the joints can form the subject of a statistical analysis allowing to monitor the progression and the performance of the operator. After an intervention, it is, for example, possible to replay a video representing the movements made by the operator, on the computer screen displaying the avatar, plus the triggering of possible alerts. Thus, the operator is able to perfect their practice.

Furthermore, according to an advanced embodiment, in order to prevent the appearance of MSD, the present invention provides the use of data from angle measurements on at least two joints of a limb.

Indeed, preventing MSDs consisting only of training users to individually hold each one of the joints applied for a task, in an angle called “comfort”, such that said task is the least traumatic as possible for the articular and periarticular anatomic structures (mainly the tendon which holds the muscle by insertion on the bone), can prove to be non-optimal.

Indeed, this “univalent” approach of the cause of the appearance of periarticular lesions does not consider the muscular synergy occurring on the close joints when making a movement. For example, if the prevention settles for holding the elbow joint in the “comfort” angle zone when bending the forearm over the arm, which mainly brings into play the bicep, it does not consider the contraction of the deltoid muscle. This, although the shoulder joint can be in a “comfort” angle, will act on the periarticular elements of the rotator cuff, and, when this movement is repeated at the level of the elbow, lesions can appear at the level of the shoulder.

Because of this, it is preferred, according to the invention, that the prevention of periarticular diseases, overall, aims for all joints of a limb, or at least the joint or joints close to the joint making the movement.

To this end, the present invention also aims for a method, advantageously based on using an active orthosis system such as defined above and a calculator. According to said method, a posture is recommended to a technician, said recommended posture aiming for a limb of said technician, for example, an upper limb, according to the angle measurements of at least two joints of said limb, said measurements being taken by means of said active orthosis system. For example, said calculator determines, from the values measured, thanks to the active orthosis, the angles formed by at least two joints of a user's limb, typically the shoulder and the elbow, the optimal position of the whole arm to limit the risk of injury, while achieving the movement desired by the user.

The invention thus relates to a method for determining an optimum between the angles of at least two joints of a limb to minimise the risk of MSD, according to the angles of said at least two joints measured by means of the active orthosis defined above.

It must be noted that the active orthosis system according to the invention is not limited to the embodiments defined and can form the subject of alternatives in the scope of a person skilled in the art.

As already stated, the active orthosis system according to the invention, is particularly likely for application in many technical fields, and could not be limited to the field of movement made by animal insemination technicians.

While the present disclosure has been illustrated and described with respect to a particular embodiment thereof, it should be appreciated by those of ordinary skill in the art that various modifications to this disclosure may be made without departing from the spirit and scope of the present disclosure.

What is claimed is:

1. An active orthosis system, comprising a plurality of inertial sensors configured to be distributed over an upper limb, a lower limb, or any other part of the human body comprising at least one joint, wherein said inertial sensors are designed to allow the determination of at least one angle formed by segments of said part of the human body, around said at least one joint, and wherein the active orthosis system comprises an alert device to warn the user of the active orthosis system that said at least one angle has a value located outside of a predetermined range of comfort values.

2. The active orthosis system according to claim 1, comprising software means for representing on a computer screen at least said part of the human body, in the form of an avatar, presenting, in real time, said at least one angle of said at least one joint, such as determined.

3. The active orthosis system according to claim 1, comprising means for presenting to the user, visual and/or sound information adapter to lead them to correct their posture so as to hold said at least one angle in the predetermined range of comfort values.

4. The active orthosis system according to claim 3, comprising means for generating vibrations felt by the user when said at least one angle passes outside of the predetermined range of comfort values.

5. The active orthosis system according to claim 1, comprising wireless communication means, for example, conforming with the Bluetooth standard, so as to communicate data from the plurality of sensors to a calculator or so as to communicate information to the user.

6. The active orthosis system according to claim 1, wherein the plurality of inertial sensors comprises at least one sensor of at least one of the following types: accelerometer; gyroscope; magnetometer.

7. The active orthosis system according to claim 1, wherein each inertial sensor of the plurality of inertial sensors consists of one detection module, nine axes comprising a three-axis accelerometer, a three-axis gyroscope and a three-axis magnetometer.

8. The active orthosis system according to claim 1, comprising a microcontroller.

9. The active orthosis system according to claim 1, wherein the part of the human body is an upper limb, the active orthosis system comprising four inertial sensors arranged respectively on the middle of the arm, on the middle of the forearm, on the back of the hand and at the level of the plexus, allowing to determine the angles of the shoulder, the elbow and the wrist.

10. The active orthosis system according to claim 1, comprising software means capable of transferring a set of parameters to the active orthosis system, as well as collecting and processing information from the plurality of inertial sensors statistically.

11. A method for determining a recommended value for at least two angles corresponding to at least two joints of a part of the human body, such as an upper limb, according to the determination, by an active orthosis system according to claim 1, of said at least two angles, formed by segments of said part of the human body, respectively around each one of said at least two joints.

12. The method according to claim 11, wherein the active orthosis system comprises software means for representing on a computer screen at least said part of the human body, in the form of an avatar, presenting, in real time, said at least one angle of said at least one joint, such as determined.

13. The method according to claim 11, wherein the active orthosis system comprises means for presenting to the user, visual and/or sound information adapter to lead them to correct their posture so as to hold said at least one angle in the predetermined range of comfort values.

14. The method according to claim 13, wherein the active orthosis system comprises means for generating vibrations felt by the user when said at least one angle passes outside of the predetermined range of comfort values.

15. The method according to claim 11, wherein the active orthosis system comprises wireless communication means, for example, conforming with the Bluetooth standard, so as to communicate data from the plurality of sensors to a calculator or so as to communicate information to the user.

16. The method according to claim 11, wherein the plurality of inertial sensors comprises at least one sensor of at least one of the following types: accelerometer; gyroscope; magnetometer.

17. The method according to claim 11, wherein each inertial sensor of the plurality of inertial sensors consists of one detection module, nine axes comprising a three-axis accelerometer, a three-axis gyroscope and a three-axis magnetometer.

18. The method according to claim 11, wherein the active orthosis system comprises a microcontroller.

19. The method according to claim 11, wherein the part of the human body is an upper limb, the active orthosis system comprising four inertial sensors arranged respectively on the middle of the arm, on the middle of the forearm, on the back of the hand and at the level of the plexus, allowing to determine the angles of the shoulder, the elbow and the wrist.

20. The method according to claim 11, wherein the active orthosis system comprises software means capable of transferring a set of parameters to the active orthosis system, as well as collecting and processing information from the plurality of inertial sensors statistically.
